(19) United States
(12) Patent Application Publication
(10) Pub. No.: US 2024/0032941 A1
(43) Pub. Date: Feb. 1, 2024

(54) EMBOLIC MATERIAL DELIVERY DEVICE AND RELATED TECHNOLOGY

(71) Applicant: Covidien LP, Mansfield, MA (US)

(72) Inventors: Jared Akira Shimizu, Tustin, CA (US); Junwei Li, Irvine, CA (US); Madeleine Roseen, Irvine, CA (US); Mehdi Matteo Rashidi, Irvine, CA (US)

(21) Appl. No.: 17/815,677

(22) Filed: Jul. 28, 2022

(51) Int. Cl. A61B 17/12

(52) U.S. Cl. CPC A61B 17/12186; A61B 17/12113; A61B 2017/00867

(57) ABSTRACT

A device for treating an intracranial aneurysm in accordance with an embodiment of the present technology includes an elongate conduit body defining an axial lumen through which the conduit body is configured to convey liquid embolic material toward the aneurysm. The conduit body includes a first flexibility zone defining a first average bending stiffness and a first outer diameter. The conduit body further includes a second flexibility zone distal to the first flexibility zone and defining a second average bending stiffness and a second outer diameter. The second average bending stiffness and the second outer diameter are less than the first average bending stiffness and the first outer diameter, respectively. The conduit body also includes a transition zone between the first and second flexibility zones. The transition zone defines a third outer diameter that transitions proximally-to-distally from the first outer diameter to the second outer diameter.

### TECHNICAL FIELD

The present technology relates to devices for treating aneurysms.

### BACKGROUND

An aneurysm is an abnormal bulging or ballooning at a weakening in a wall of a blood vessel. Causes of aneurysms include disease, injury, and congenital abnormality. Although aneurysms can occur in many different parts of the body, the most common locations are the aorta and the intracranial vasculature. It is estimated that 2% or more of the worldwide population harbors an unruptured intracranial aneurysm. Many of these intracranial aneurysms eventually rupture leading to severe complications, such as subarachnoid hemorrhage. Unfortunately, the prognosis for subarachnoid hemorrhage is poor. Most patients with this condition either die or suffer from long-term cognitive impairment. The probability of death or disability from a ruptured aortic aneurysm can be even higher than from a ruptured intracranial aneurysm. Fortunately, treatments for ruptured and unruptured aneurysms currently exist and continue to improve. Many of these treatments involve reducing blood flow within an aneurysm and thereby promoting thrombosis and embolization. Aneurysms treated in this manner are significantly less likely to rupture than untreated aneurysms. These and other treatments have the potential to save thousands of lives every year. Accordingly, there is an ongoing public health need for inventive effort in this field.

### SUMMARY

An embolic material delivery device in accordance with at least some embodiments of the present technology includes a conduit body extending between a proximal end portion and a distal end portion and defining an axial lumen also extending between these portions. The axial lumen is configured to transport embolic material. The conduit body comprises a first flexibility zone, a transition zone, and a second flexibility zone. The first flexibility zone is positioned between the proximal end portion and the transition zone and is configured to transfer pushing force toward the second flexibility zone. The second flexibility zone is positioned between the transition zone and the distal end portion and is configured to be navigable through tortuous neurovascular anatomy. The first and second flexibility zones define first and second kink radiuses, respectively, with the second kink being smaller than the first kink radius.

A device in accordance with at least some embodiments of the present technology is suitable for treating an intracranial aneurysm. The device comprises an elongate conduit body configured to extend intravascularly toward the aneurysm and defining an axial lumen through which the conduit body is configured to convey liquid embolic material toward the aneurysm. The conduit body comprises a proximal end portion and a distal end portion opposite to the proximal end portion along a length of the conduit body. The conduit body further comprises a first flexibility zone defining a first portion of the length of the conduit body, a second flexibility zone defining a second portion of the length of the conduit body distal to the first portion of the length of the conduit body, and a transition zone defining a third portion of the length of the conduit body between the first and second portions of the length of the conduit body. The first and second flexibility zones further define first and second average bending stiffnesses, respectively, with the second average bending stiffness being less than the first average bending stiffness. The first and second flexibility zones still further define first and second outer diameters, respectively, with the second outer diameter being less than the first outer diameter. The transition zone further defines a third outer diameter that transitions proximally-to-distally from the first outer diameter to the second outer diameter. The device further comprises an expandable structure carried by the conduit body. The expandable structure is configured to be disposed at least partially within the aneurysm to reduce leakage of liquid embolic material from the aneurysm. The device also comprises a detachment element through which the expandable structure is detachably connected to the conduit body. The detachment element is configured to detach the expandable structure from the conduit body such that the conduit body can be withdrawn intravascularly away from the aneurysm while the expandable structure remains disposed at least partially within the aneurysm.

A method in accordance with at least some embodiments of the present technology is suitable for treating an aneurysm at a neurovascular treatment location. The method comprises moving a distal end portion of an elongate conduit body intravascularly toward the treatment location. The method further comprises flowing liquid embolic material defining a viscosity greater than 30 centistokes distally within a first portion of an axial lumen defined by the conduit body. The method still further comprises flowing the liquid embolic material distally within a second portion of the axial lumen distal to the first portion of the axial lumen. The method also comprises flowing the liquid embolic material into the aneurysm. The first and second portions of the axial lumen define first and second pressure drops per unit length, respectively, with the second pressure drop per unit length being at least 10% less than the first pressure drop per unit length.

Examples of aspects of the present technology are described below as numbered clauses (1, 2, 3, etc.) for convenience. These are provided as examples and do not limit the present technology.
Clause 1. An embolic material delivery device comprising:
a conduit body extending between a proximal end portion and a distal end portion, wherein the conduit body defines an axial lumen extending between the proximal end portion and the distal end portion, and wherein the axial lumen is configured to transport the embolic material,
wherein the conduit body comprises a first flexibility zone, a transition zone, and a second flexibility zone,
wherein the first flexibility zone is positioned between the proximal end portion and the transition zone, and wherein the second flexibility zone is positioned between the transition zone and the distal end portion, and
wherein the first flexibility zone is configured to transfer pushing force toward the second flexibility zone and defines a first kink radius, and wherein the second flexibility zone is configured to be navigable through tortuous neurovascular anatomy and defines a second kink radius smaller than the first kink radius.
Clause 2. The embolic material delivery device of any of the preceding or following clauses, wherein the second kink radius is within a range from 10% to 80% of the first kink radius.
Clause 3. The embolic material delivery device of any of the preceding or following clauses, further comprising:
a first tube defining a first outer diameter at the first flexibility zone; and
a second tube defining a second outer diameter at the second flexibility zone, wherein the second outer diameter is less than the first outer diameter, and wherein the first tube and the second tube are coupled to one another at the transition zone.
Clause 4. The embolic material delivery device of any of the preceding or following clauses, further comprising a welded joint coupling the first tube and the second tube to one another at the transition zone.
Clause 5. The embolic material delivery device of any of the preceding or following clauses, wherein the second tube is positioned within the first tube at the transition zone.
Clause 6. The embolic material delivery device of any of the preceding or following clauses, wherein the first tube and the second tube are formed from the same material.
Clause 7. The embolic material delivery device of any of the preceding or following clauses, wherein the second tube is annealed and the first tube is not annealed or is annealed to a lesser extent than the second tube.
Clause 8. The embolic material delivery device of any of the preceding or following clauses, wherein the first tube and the second tube are nitinol.
Clause 9. The embolic material delivery device of any of the preceding or following clauses, wherein the first flexibility zone, the second flexibility zone, and the transition zone are a monolithic structure formed from a single piece of material.
Clause 10. The embolic material delivery device of any of the preceding or following clauses, wherein:
the first flexibility zone defines a first outer diameter; and
the second flexibility zone defines a second outer diameter less than the first outer diameter.
Clause 11. The embolic material delivery device of any of the preceding or following clauses, wherein the conduit body is formed from a tube defining a constant outer diameter and the second flexibility zone is formed by removing material from the tube in order to reduce the outer diameter of a portion of the tube to the second outer diameter.
Clause 12. The embolic material delivery device of any of the preceding or following clauses, wherein the transition zone defines a tapering outer diameter transitioning from the first outer diameter to the second outer diameter.
Clause 13. The embolic material delivery device of any of the preceding or following clauses, wherein the taper extends at least 2 cm in order for the conduit body to define a variable kink radius transitioning from the first kink radius to the second kink radius.
Clause 14. The embolic material delivery device of any of the preceding or following clauses, wherein the axial lumen defines a constant inner diameter.
Clause 15. The embolic material delivery device of any of the preceding or following clauses, wherein:
the transition zone is a first transition zone; and
the conduit body comprises:
   a third flexibility zone, and
   a second transition zone between the second flexibility zone and the third flexibility zone.
Clause 16. The embolic material delivery device of any of the preceding or following clauses, wherein one of the first transition zone or the second transition zone comprises overlapping tubes and a welded coupling, and the other comprises a taper between two monolithic flexibility zones.
Clause 17. The embolic material delivery device of any of the preceding or following clauses, further comprising a heat shrink positioned around the first flexibility zone, the transition zone, and the second flexibility zone.
Clause 18. A device for treating an intracranial aneurysm, the device comprising:
an elongate conduit body configured to extend intravascularly toward the aneurysm, wherein the conduit body defines an axial lumen through which the conduit body is configured to convey liquid embolic material toward the aneurysm, and wherein the conduit body comprises:
   a proximal end portion,
   a distal end portion opposite to the proximal end portion along a length of the conduit body,
   a first flexibility zone defining a first portion of the length of the conduit body, a first average bending stiffness, and a first outer diameter,
   a second flexibility zone defining a second portion of the length of the conduit body, a second average bending stiffness, and a second outer diameter, wherein the second portion of the length of the conduit body is distal to the first portion of the length of the conduit body, wherein the second average bending stiffness is less than the first average bending stiffness, and wherein the second outer diameter is less than the first outer diameter,
   a transition zone defining a third portion of the length of the conduit body and a third outer diameter, wherein the third portion of the length of the conduit body is between the first and second portions of the length of the conduit body, and wherein the third outer diameter transitions proximally-to-distally from the first outer diameter to the second outer diameter;
an expandable structure carried by the conduit body, wherein the expandable structure is configured to be disposed at least partially within the aneurysm to reduce leakage of liquid embolic material from the aneurysm; and
a detachment element through which the expandable structure is detachably connected to the conduit body, wherein the detachment element is configured to detach the expandable structure from the conduit body such that the conduit body can be withdrawn intravascularly away from the aneurysm while the expandable structure remains disposed at least partially within the aneurysm.
Clause 19. The device of any of the preceding or following clauses, wherein the third outer diameter steps down proximally-to-distally from the first outer diameter to the second outer diameter.
Clause 20. The device of any of the preceding or following clauses, wherein the third outer diameter tapers down proximally-to-distally from the first outer diameter to the second outer diameter.
Clause 21. The device of any of the preceding or following clauses, wherein the second average bending stiffness is within a range from 5% to 25% of the first average bending stiffness.
Clause 22. The device of any of the preceding or following clauses, wherein
the first portion of the length of the conduit body is at least 5 cm;
the first outer diameter varies no more than 5% along the first portion of the length of the conduit body;
the second portion of the length of the conduit body is at least 5 cm; and
the second outer diameter varies no more than 5% along the second portion of the length of the conduit body.
Clause 23. The device of any of the preceding or following clauses, further comprising:
an inlet port operably connected to the axial lumen, wherein the device is configured to receive liquid embolic material via the inlet port, and wherein the transition zone is at least 20 cm distal to the inlet port; and
an outlet port operably connected to the axial lumen, wherein the device is configured to dispense liquid embolic material into the aneurysm via the outlet port, and wherein the transition zone is and at least 20 cm proximal to the outlet port along the length of the conduit body.
Clause 24. The device of any of the preceding or following clauses, wherein:
the conduit body comprises a tube extending along the first, second, and third portions of the length of the conduit body and defining the first flexibility zone, the second flexibility zone, and the transition zone;
the first flexibility zone, the second flexibility zone, and the transition zone define a first wall thickness, a second wall thickness, and a third wall thickness, respectively;
the first wall thickness is greater than the second wall thickness; and
the third wall thickness decreases proximally-to-distally from the first wall thickness to the second wall thickness.
Clause 25. The device of any of the preceding or following clauses, wherein:
the first flexibility zone, the second flexibility zone, and the transition zone define a first inner diameter, a second inner diameter, and a third inner diameter, respectively; and
the first, second, and third inner diameters are equal.
Clause 26. The device of any of the preceding or following clauses, wherein the first flexibility zone defines a pressure rating of at least 20 kpsi.
Clause 27. The device of any of the preceding or following clauses, wherein:
the conduit body comprises:
   a first tube extending along the first and third portions of the length of the conduit body, and
   a second tube extending along the second and third portions of the length of the conduit body; and
the second tube overlaps the first tube at the transition zone.
Clause 28. The device of any of the preceding or following clauses, wherein the first tube comprises a first material, and wherein the second tube comprises a second material compositionally the same as and microstructurally different than the first material.
Clause 29. The device of any of the preceding or following clauses, wherein:
the first flexibility zone, the second flexibility zone, and the transition zone define a first inner diameter, a second inner diameter, and a third inner diameter, respectively;
the first inner diameter is greater than the second inner diameter; and
the second and third inner diameters are equal.
Clause 30. A method for treating an aneurysm at a neurovascular treatment location, the method comprising:
moving a distal end portion of an elongate conduit body intravascularly toward the treatment location, wherein the conduit body defines an axial lumen;
flowing liquid embolic material defining a viscosity greater than 30 centistokes distally within a first portion of the axial lumen, wherein the first portion of the axial lumen defines a first pressure drop per unit length;
flowing the liquid embolic material distally within a second portion of the axial lumen distal to the first portion of the axial lumen, wherein the second portion of the axial lumen defines a second pressure drop per unit length at least 10% less than the first pressure drop per unit length; and
flowing the liquid embolic material into the aneurysm.
Clause 31. The method of any of the preceding or following clauses, further comprising locating an expandable structure carried by the conduit body at least partially within the aneurysm to reduce leakage of the liquid embolic material from the aneurysm, wherein flowing the liquid embolic material into the aneurysm causes the expandable structure to transition from a first state toward a second state, and wherein the expandable structure occupies less space within the aneurysm in the second state than in the first state.
Clause 32. The method of any of the preceding or following clauses, wherein moving the distal end portion intravascularly toward the treatment location comprises bending a second flexibility zone of the conduit body to a radius smaller than a kink radius of a first flexibility zone of the conduit body, wherein the first flexibility zone defines the first portion of the axial lumen, and wherein the second flexibility zone defines the second portion of the axial lumen.

### BRIEF DESCRIPTION OF THE DRAWINGS

Many aspects of the present technology can be better understood with reference to the following drawings. The relative dimensions in the drawings may be to scale with respect to some embodiments of the present technology. With respect to other embodiments, the drawings may not be to scale. The drawings may also be enlarged arbitrarily. For clarity, reference-number labels for analogous components or features may be omitted when the appropriate reference-number labels for such analogous components or features are clear in the context of the specification and all of the drawings considered together. Furthermore, the same reference numbers may be used to identify analogous components or features in multiple described embodiments.

FIG. 1 is a profile view of an embolic material delivery device in accordance with at least some embodiments of the present technology.

FIG. 2 is an end view of the embolic material delivery device shown in FIG. 1.

FIG. 3 is a longitudinal cross-sectional profile view of the embolic material delivery device shown in FIG. 1 taken along the line A-A in FIG. 2.

FIG. 4 is an enlarged view of a portion of FIG. 3 showing a transition zone and neighboring portions of the embolic material delivery device shown in FIG. 1.

FIGS. 5-7 are longitudinal cross-sectional views corresponding to FIG. 4 showing alternative transition zones and neighboring portions of embolic material delivery devices in accordance with at least some embodiments of the present technology.

FIG. 8 is a profile view of an embolic material delivery device in accordance with at least some embodiments of the present technology.

FIG. 9 is a longitudinal cross-sectional view of an embolic material delivery device in accordance with at least some embodiments of the present technology.

FIG. 10 is a perspective view of the embolic material delivery device shown in FIG. 1 and associated structures within a system in accordance with at least some embodiments of the present technology.

FIGS. 11-15 are longitudinal cross-sectional views of portions of the system shown in FIG. 10 and relevant anatomy at different respective times during a method for treating an aneurysm at a neurovascular treatment location in accordance with at least some embodiments of the present technology.

### DETAILED DESCRIPTION

Disclosed herein are examples of embolic material delivery devices and related technology. As discussed above, treatment of an aneurysm can involve reducing blood flow within the aneurysm and thereby promoting thrombosis and embolization. One approach to reducing blood flow within an aneurysm includes introducing a liquid embolic material into the aneurysm. A conduit body can be advanced intravascularly to establish a flow channel between an extracorporeal inlet port and an outlet port within the aneurysm. Liquid embolic material can then be flowed through this channel under pressure to reach the aneurysm. In at least some cases, the conduit body also carries an expandable structure that is also introduced into the aneurysm. As the liquid embolic material fills the aneurysm, the expandable structure can collapse against the neck of the aneurysm to reduce or prevent leakage of the liquid embolic material. Once the aneurysm is sufficiently filled, the conduit body can be detached from the expandable structure and removed.

One challenge associated with embolic material delivery devices is that liquid embolic material is often very viscus. High-viscosity liquid embolic materials are preferred for certain procedures because they tend to flow in a more controlled manner than low-viscosity embolic materials and/or for other reasons. The flow channel for delivery of liquid embolic material to an intracranial aneurysm, however, is typically long and narrow such that achieving even a reasonable flowrate of high-viscosity embolic material necessitates the use of high pressures. The need to withstand these high pressures can limit the design and materials of conduit bodies in ways that are contrary to promoting other desirable functionality. Conduit bodies that are stiffer tend to be better at withstanding high pressures and easier to push. A relatively stiff conduit body, however, tends to be less able than a more flexible conduit body to move through tortuous anatomy. For example, force from blood vessel walls may be insufficient to cause a relatively stiff conduit body to curve as needed while the conduit body advances through the blood vessel. In addition or alternatively, a relatively stiff conduit body may tend to kink rather than flex in response to this curving. Kinking can block or narrow the flow channel thereby preventing or inhibiting the flow of embolic material.

Embolic material delivery devices in accordance with embodiments of the present technology at least partially address one or more of the foregoing challenges and/or other challenges associated with conventional technologies. In a particular example, an embolic material delivery device in accordance with at least some embodiments of the present technology includes a conduit body with different flexibility zones at different portions of its length. The inventors recognized that design tradeoffs for a conduit body are different at different portions of the length of the conduit body. For example, a more proximal portion of the conduit body can include features that reduce a pressure drop of the flow channel because this portion is less likely than a more distal portion of the conduit body to encounter narrow vasculature that would limit the potential size of the flow channel. Similarly, the proximal portion of the conduit body can include features that increase a resistance of the conduit body to high pressures because the pressure of liquid embolic material decreases as it moves distally during delivery. Also, the proximal portion of the conduit body can include features that facilitate handling because this is the portion that is subject to handling and/or features that increase pushability because the need for pushability decreases proximally to distally along the length of the conduit body. In contrast, a more distal portion of the conduit body can include features that promote flexibility and/or kink resistance because this portion is more likely than the proximal portion to encounter small and tortuous vasculature during delivery. Variation between the proximal and distal portions of the conduit body can be in outer diameter, inner diameter, wall thickness, material, degree of annealing, type of annealing, and/or one or more other properties. Intermediate portions of the conduit body can include facilitate transitions in one or more of these properties.

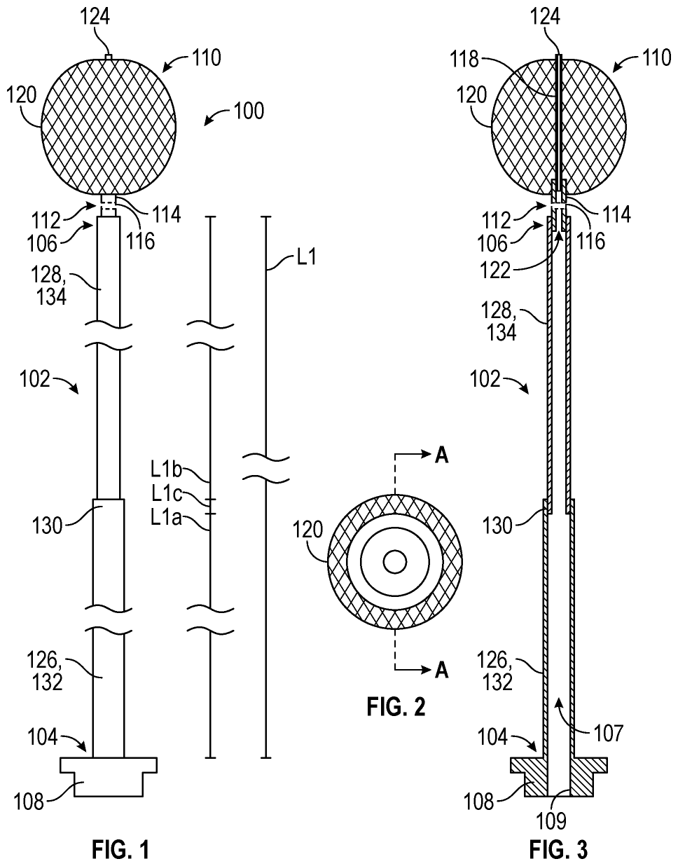

Specific details of systems, devices, and methods for treating intracranial aneurysms in accordance with embodiments of the present technology are described herein with reference to FIGS. 1-15. Although these systems, devices, and methods may be described herein primarily or entirely in the context of treating saccular intracranial aneurysms, other contexts are within the scope of the present technology. For example, suitable features of described systems, devices, and methods for treating saccular intracranial aneurysms can be implemented in the context of treating non-saccular intracranial aneurysms, abdominal aortic aneurysms, thoracic aortic aneurysms, renal artery aneurysms, arteriovenous malformations, tumors (e.g. via occlusion of vessel(s) feeding a tumor), perivascular leaks, varicose veins (e.g. via occlusion of one or more truncal veins such as the great saphenous vein), hemorrhoids, and sealing endoleaks adjacent to artificial heart valves, covered stents, and abdominal aortic aneurysm devices among other examples. Furthermore, it should be understood, in general, that other systems, devices, and methods in addition to those disclosed herein are within the scope of the present disclosure. For example, systems, devices, and methods in accordance with embodiments of the present technology can have different and/or additional configurations, components, procedures, etc. than those disclosed herein. Moreover, systems, devices, and methods in accordance with embodiments of the present disclosure can be without one or more of the configurations, components, procedures, etc. disclosed herein without deviating from the present technology.

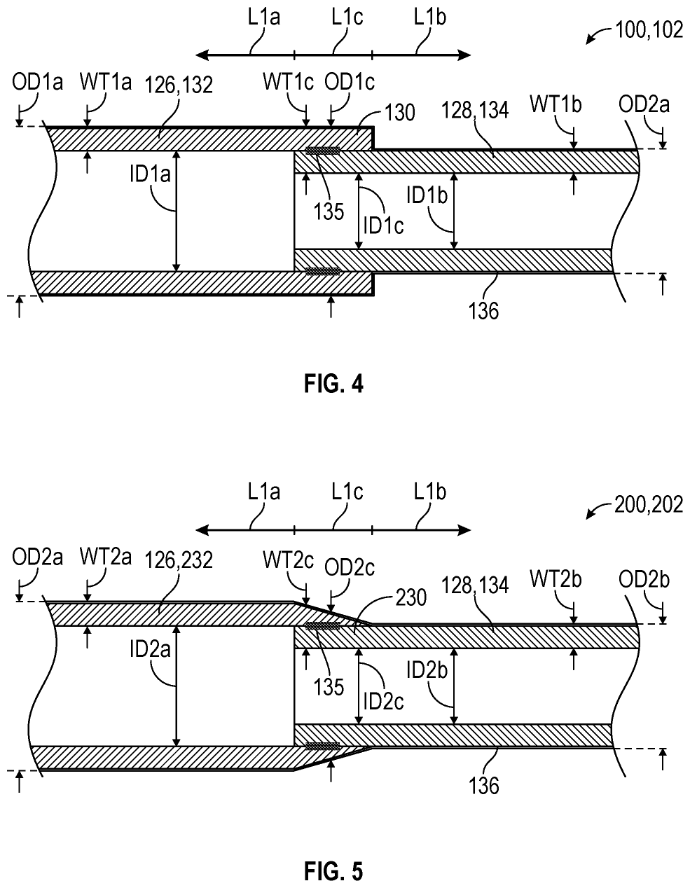

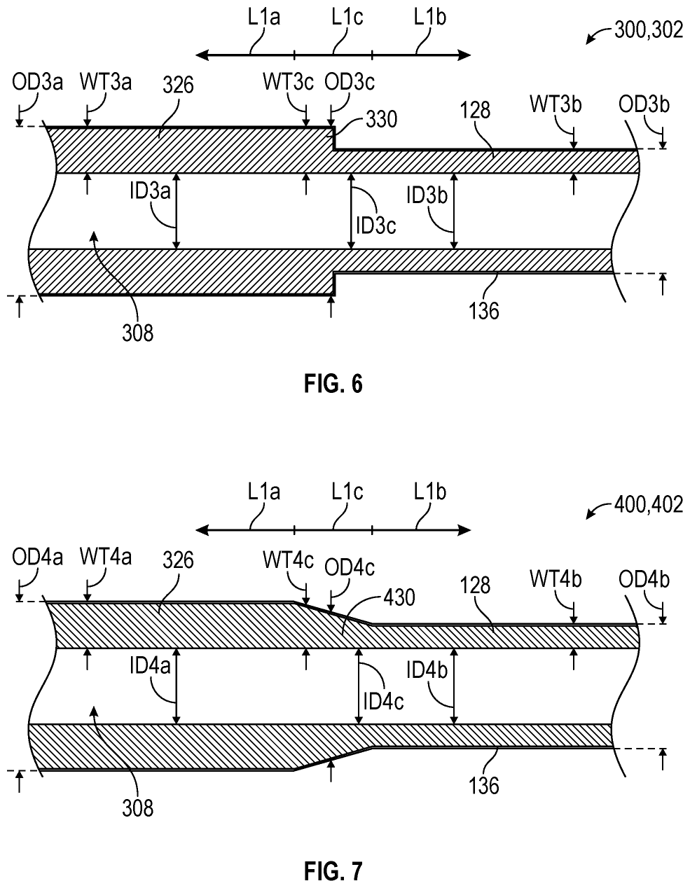

FIGS. 1 and 2 are a profile view and an end view, respectively, of an embolic material delivery device 100 in accordance with at least some embodiments of the present technology. FIG. 3 is a longitudinal cross-sectional profile view of the embolic material delivery device 100 taken along the line A-A in FIG. 2. With reference to FIGS. 1-3 together, the embolic material delivery device 100 can include an elongate conduit body 102 comprising a proximal end portion 104 and a distal end portion 106 opposite to the proximal end portion 104 along a length L1 of the conduit body 102. In at least some embodiments, the length L1 is within a range from 100 to 200 cm, such as from 125 to 175 cm. The conduit body 102 can define an axial lumen 107 extending between the proximal end portion 104 and the distal end portion 106. The embolic material delivery device 100 can be configured for treating an intracranial aneurysm. For example, the conduit body 102 can be configured to extend intravascularly toward an intracranial aneurysm and to convey liquid embolic material toward the aneurysm via the axial lumen 107. At the proximal end portion 104, the embolic material delivery device 100 can include a fitting 108 configured to be attached to a source of liquid embolic material (not shown). Also at the proximal end portion 104, the embolic material delivery device 100 can include an inlet port 109 operably connected to the axial lumen 107. The embolic material delivery device 100 can be configured to receive liquid embolic material via the inlet port 109.

The embolic material delivery device 100 can further include an expandable structure 110 configured to be disposed at least partially within the aneurysm to reduce leakage of liquid embolic material from the aneurysm. For example, the conduit body 102 can carry the expandable structure 110 at the distal end portion 106. Relatedly, the embolic material delivery device 100 can include a detachment element 112 through which the expandable structure 110 is detachably connected to the conduit body 102. The detachment element can be coupled to the distal end portion 106 of the conduit body 102 with a weld (not shown) or in another suitable manner. In at least some cases, the detachment element 112 includes a first conduit 114 including a detachment zone 116 configured to be selectively severed. In this or another manner, the detachment element 112 can be configured to detach the expandable structure 110 from the conduit body 102 such that the conduit body 102 can be withdrawn intravascularly away from an aneurysm while the expandable structure 110 remains disposed at least partially within the aneurysm. In at least some cases, the detachment element 112 comprises a nickel, cobalt, chromium, and molybdenum alloy at the detachment zone 116. In these and other cases, the detachment zone 116 can be electrically connected via the conduit body 102 to a controller (not shown) configured to be extra-corporally positioned during a treatment procedure using the embolic material delivery device 100.

The expandable structure 110 can include a second conduit 118 extending distally from the first conduit 114 and a mesh 120 fixedly connected to the first conduit 114 and slidably connected to the second conduit 118. The first and second conduits 114, 118 can define a channel 122 operably connected to the axial lumen 107. At a distal tip of the second conduit 118, the embolic material delivery device 100 can include an outlet port 124 operably connected to the axial lumen 107 via the channel 122. For example, the axial lumen 107 can be configured to transport liquid embolic material distally toward the channel 122, the channel 122 can be configured to transport liquid embolic material distally toward the outlet port 124, and the embolic material delivery device 100 can be configured to dispense liquid embolic material into an aneurysm via the outlet port 124.

The conduit body 102 can include a plurality of zones along the length L1 each with one or more different geometries, relative to adjacent zones, defining different properties related to features such as pushability, flexibility, strength, and pressure drop, among others. For example, the conduit body 102 can include a first flexibility zone 126 at a first portion L1a of the length L1, a second flexibility zone 128 at a second portion L1b of the length L1, and a transition zone 130 at a third portion L1c of the length L1 between the first and second portions L1a, L1b of the length L1. The first flexibility zone 126, the transition zone 130, and the second flexibility zone 128 can be arranged proximal-to-distal in series along the length L1. In the illustrated embodiment, these zones are directly adjacent to one another. In other embodiments, counterpart zones can be spaced apart from one another, such as with one or more intervening zones. Furthermore, one or both of the first and second portions L1a, L1b of the length L1 can be at least 3 cm, at least 5 cm, and/or at least 10 cm. In addition or alternatively, the transition zone 130 can be at least 20 cm distal to the inlet port 109 and/or at least 20 cm proximal to the outlet port 124 along the length L1. In at least some cases, the transition zone 130 is from 20 cm to 30 cm proximal to the outlet port 124 along the length L1. These and other geometries disclosed herein are merely examples. Accordingly, while the forgoing lengths may be suitable for certain neurovascular applications, other lengths may be suitable for other applications.

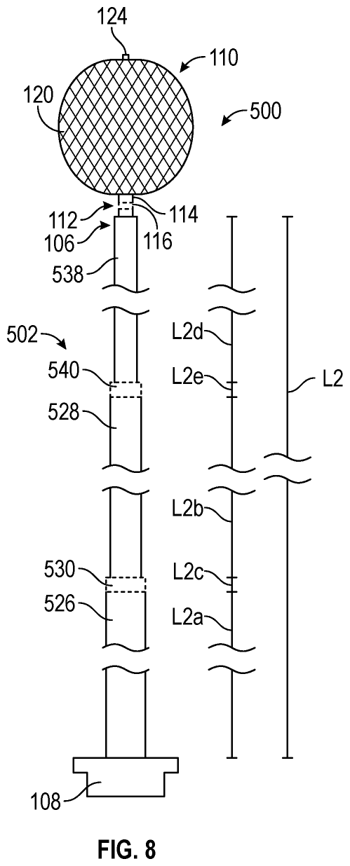

FIG. 4 is an enlarged view of a portion of FIG. 3 showing the transition zone 130 and neighboring portions of the first and second flexibility zones 126, 128. With reference to FIGS. 1-4 together, the conduit body 102 can include a first tube 132 extending along the first and third portions L1a, L1c of the length L1. The conduit body 102 can further include a second tube 134 extending along the second and third portions L1b, L1c of the length L1. In the illustrated embodiment, the first and second tubes 132, 134 are separately formed structures joined at the transition zone 130. For example, the second tube 134 can be positioned within the first tube 132 at the transition zone 130. Accordingly, the second tube 134 can overlap the first tube 132 at the transition zone 130. In addition or alternatively, the conduit body 102 can include a welded joint 135 coupling the first tube 132 and the second tube 134 to one another at the transition zone 130. The welded joint 135 can be formed thermally, adhesively, and/or in another suitable manner. The length of the overlap can define the third portion L1c of the length L1. When the third portion L1c of the length L1 is longer, the strength of a connection between the first and second tubes 132, 134 may tend to be greater than when the third portion L1c of the length L1 is shorter. Corresponding, when the third portion L1c of the length L1 is shorter, the degree to which the transition zone 130 adversely affects the flexibility of the conduit body 102 (e.g., by creating a relatively inflexible area and/or by creating stress points distal and proximal to that area) may tend to be less than when the third portion L1c of the length L1 is longer. Based on these and/or other considerations, the third portion L1c of the length L1 can be within a range from 0.25 mm to 2 mm, such as within a range from 0.25 to 1 mm.

As shown in FIG. 4, the first flexibility zone 126 and the first tube 132 can define a first outer diameter OD1a, a first inner diameter ID1a, and a first wall thickness WT1a. In at least some cases, the first outer diameter OD1a is relatively consistent (e.g., varies no more than 5%) along the first portion L1a of the length L1. The second flexibility zone 128 and the second tube 134 can define a second outer diameter OD1b, a second inner diameter ID1b, and a second wall thickness WT1b. In at least some cases, the second outer diameter OD1b is relatively consistent (e.g., varies no more than 5%) along the second portion L1b of the length L1. The transition zone 130 can define a third outer diameter OD1c, a third inner diameter ID1c, and a third wall thickness WT1c. In the illustrated embodiment, the first outer diameter OD1a is greater than the second outer diameter OD1b and equal to the third outer diameter OD1c, the second inner diameter ID1b is less than the first inner diameter ID1a and equal to the third inner diameter ID1c, the first and second wall thicknesses WT1a, WT1b are equal and less than the third wall thickness WT1c. The third wall thickness WT1c can be equal to the sum of the first and second wall thicknesses WT1a, WT1b. Alternatively, the third wall thickness WT1c can be equal to the sum of the first and second wall thicknesses WT1a, WT1b and the thickness of the welded joint 135 between portions of the first and second tubes 132, 134 that overlap at the transition zone 130. Also in the illustrated embodiment, the third outer diameter OD1c transitions proximally-to-distally from the first outer diameter OD1a to the second outer diameter OD1b. The transition can be a step down at a distal end of the transition zone 130 as shown or have another suitable form as discussed below.

With reference again to FIG. 4, the embolic material delivery device 100 can further include a heat shrink 136 positioned around the first flexibility zone 126, the transition zone 130, and the second flexibility zone 128. The heat shrink 136 can be useful, for example, to reduce friction as the conduit body 102 moves through vasculature. This can be particularly useful at the transition zone 130 where a change in the third outer diameter OD1c may tend to increase friction. Alternatively or in addition, the heat shrink 136 can be useful to electrically insulate portions of the conduit body 102. For example, all but the proximal end portion 104 of the conduit body 102 can be electrically insulated such that an electrical current can be passed from the proximal end portion 104 to the detachment zone 116. This electrical current can be used to selectively sever the detachment element 112 at the detachment zone 116. Suitable materials for the heat shrink 136 include polytetrafluoroethylene and polyimides.

The first flexibility zone 126 can be configured to transfer pushing force toward the second flexibility zone 128. The second flexibility zone 128 can be configured to be navigable through tortuous neurovascular anatomy. These and other functional features of the first and second flexibility zones 126, 128 can be related to the dimensions described above and/or the relationships between these dimensions. For example, at least some of these dimensions can correspond to a desirable difference in the properties of the conduit body 102 at different portions of the length L1. In some cases, the difference in the first and second inner diameters ID1a, ID1b can correspond to a difference in free-passage area and pressure drop per unit length between the first and second flexibility zones 126, 128. For example, the first flexibility zone 126 can define a first portion of the axial lumen 107 that in turn defines a first pressure drop per unit length. The second flexibility zone 128 can define a second portion of the axial lumen 107 that in turn defines a second pressure drop per unit length less than the first pressure drop per unit length, such as at least 10%, 20% or 30% less than the first pressure drop per unit length. This can be useful, for example, when reducing an overall pressure drop of the axial lumen 107 is often desirable because more proximal portions of the conduit body 102 have less need than more distal portions of the conduit body 102 to be small enough to travel through narrow vasculature. In these and other cases, space constraints of the distal vasculature need not dictate a free passage area throughout the axial lumen 107.

In addition to or instead of differences in dimensions, the first and second tubes 132, 134 can be composed of different materials and/or be formed with different manufacturing processes. Furthermore, when the first and second tubes 132, 134 are replaced with a single tube, counterparts of the first and second flexibility zones 126, 128 as different portions of that tube can be made of different materials and/or be made with different processes. With reference to the illustrated embodiment, the first tube 132 and the first flexibility zone 126 can comprise a first material and the second tube 134 and the second flexibility zone 128 can comprise a second material. The first and second materials can be the same or compositionally and/or microstructurally different. In some cases, the first and second material are nitinol. In other cases, the first and second material are stainless steel. In still other cases, the first material is nitinol and the second material is stainless steel. In still other cases, the first material is stainless steel and the second material is nitinol. Having the first and second materials be the same, however, can be advantageous to reduce or prevent galvanic corrosion. In any of the forgoing and other cases, the first material can be not annealed and second material can be annealed. Alternatively, both the first and second materials can be annealed to different degrees, for example, with the first material being annealed to a lesser extent than the second material. Annealing is a heat treatment that typically increases ductility and decreases hardness.

Due to differences in dimensions, materials, processing, and/or for one or more other reasons, the first flexibility zone 126 can define a first kink radius and the second flexibility zone can define a second kink radius smaller than the first kink radius. In some embodiments, the second kink radius is no more than 80%, 50% or 30% of the first kink radius. In addition or alternatively, the second kink radius can be within a range from 10% to 80% or from 10% to 50% of the first kink radius. As used herein “kink radius” means the radius at which a structure will permanently deform or break. A structure with a large kink radius (e.g., a wood beam) will permanently deform or break when it is bent only a small amount, whereas a structure with a small kink radius (e.g., a flexible cable) can be bent much more significantly before deforming or breaking. For most purposes, a small kink radius is desirable in a structure used intravascularly. A large kink radius, however, may be acceptable in association with other desirable properties (e.g., pushability and strength) in portions of such a structure that do not tend to encounter tortuosity. In at least some cases, the first flexibility zone 126 defines a kink radius less than 0.25 inches, such as within a range from 0.05 to 0.15 inches. In these and other cases, the second flexibility zone 128 can define a kink radius greater than 0.5 inch, such as within a range from 0.5 to 1 inch.

Similar to kink radius, the first flexibility zone 126 can define a first bending stiffness and the second flexibility zone can define a second bending stiffness less than the first bending stiffness. As used herein “bending stiffness” means the amount force needed to bend a structure in a direction. Low bending force is typically desirable for structures used within the brain where the tortuosity of the vessels tends to be high. If an intravascular structure used within the brain does not bend easily, it may not be flexible enough to move through tortuous vessels at all or it may tend to straighten out the vessels which can cause clinical complications. In contrast, is typically desirable for portions of an intravascular structure that a clinician holds to have relatively low bending stiffness. Otherwise, these portions may have insufficient column strength and avoid collapse and/or may be difficult to push.

In at least some cases, the second bending stiffness is within a range from 3% to 50% and/or within a range from 5% to 25% of the first average bending stiffness. In these and other cases, the first flexibility zone 126 can define a first pressure rating and the second flexibility zone can define a second pressure rating smaller than the first pressure rating. Furthermore, the first pressure rating can be at least 10 kpsi, 15 kpsi, 20 kpsi, or 25 kpsi. These and other relatively high pressure ratings can be useful, for example, to allow the embolic material delivery device 100 to be used with highly viscus liquid embolic materials, which, as discussed above, are preferred for some procedures. In at least some embodiments, the first flexibility zone 126, the transition zone 130, and the second flexibility zone 128 are configured for a fluid at a pressure of greater than 10 kpsi, 15 kpsi, 20 kpsi, or 25 kpsi to flow through the axial lumen 107 and/or defining a viscosity greater than 30 centistokes.

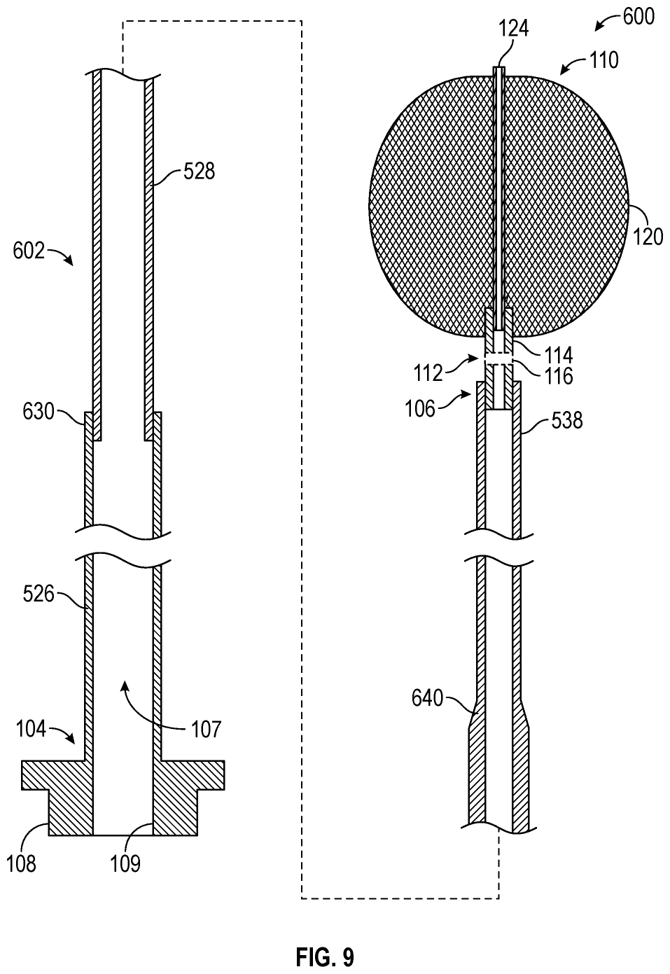

FIGS. 5-7 are longitudinal cross-sectional views corresponding to FIG. 4 showing alternative transition zones and neighboring portions of embolic material delivery devices in accordance with at least some embodiments of the present technology. For ease of reference, dimensions of the embolic material delivery devices shown in FIGS. 5-7 corresponding to the dimensions of the embolic material delivery device 100 shown in FIG. 4 are labeled with “2” as the third character in FIG. 5, with a “3” as the third character in FIG. 6, and with a “4” as the third character in FIG. 7. For example, the dimension in FIG. 5 corresponding to the first outer diameter OD1a in FIG. 4 is labeled OD2a in FIG. 5.

With reference first to FIG. 5, a conduit body 202 of an embolic material delivery device 200 in accordance with at least some embodiments of the present technology can include a transition zone 230 and a first tube 232 that differ from the transition zone 130 and the first tube 132 shown in FIG. 4. As shown in FIG. 5, the third outer diameter OD2c can taper down or otherwise decrease at least somewhat gradually proximally-to-distally from the first outer diameter OD2a to the second outer diameter OD2b. Correspondingly, the third wall thickness WT2c can taper down or otherwise decrease at least somewhat gradually proximally-to-distally from the first wall thickness WT2a to the second wall thickness WT2b. In at least some cases, the third outer diameter OD2c and the third wall thickness WT2c taper down proximally-to-distally throughout the third portion L1c of the length L1a.

With reference now to FIG. 6, a conduit body 302 of an embolic material delivery device 300 in accordance with at least some embodiments of the present technology can include a single tube extending along the first, second, and third portions L1a, L1b, L1c of the length L1 and defining a first flexibility zone 326, a transition zone 330, and a second flexibility zone 328 different than the first flexibility zone 126, the transition zone 130, and the second flexibility zone 128 shown in FIG. 4. In addition to a conduit body with transition tubes being formed from multiple tubes welded together, a conduit body 302, or a portion thereof, can be formed from a tube defining a constant outer diameter. Correspondingly, the first flexibility zone 326, the second flexibility zone 328, and the transition zone 330 can be a monolithic structure formed from a single piece of material. In these and other cases, the second flexibility zone 328 can be formed by removing material from the constant outer diameter tube in order to reduce the outer diameter of a portion of the tube to the second outer diameter OD3b. The starting tube can have a constant inner diameter such that the first, second, and third inner diameters ID3a, ID3b, ID3c are equal. In at least some of these cases, an inner diameter of the conduit body 302 is equal throughout the length L1.

In some embodiments, a transition zone of a conduit body can include a tapering profile. For example, with reference to FIG. 7, a conduit body 402 of an embolic material delivery device 400 in accordance with at least some embodiments of the present technology can be similar to the conduit body 302 shown in FIG. 6, but include a transition zone 430 that differs from the transition zone 330 shown in FIG. 6. As shown in FIG. 7, the third outer diameter OD4c can taper down or otherwise decrease at least somewhat gradually proximally-to-distally from the first outer diameter OD4a to the second outer diameter OD4b. Correspondingly, the third wall thickness WT4c can taper down or otherwise decrease at least somewhat gradually proximally-to-distally from the first wall thickness WT4a to the second wall thickness WT4b. In at least some cases, the third outer diameter OD4c and the third wall thickness WT4c taper down proximally-to-distally throughout the third portion L1c of the length L1a. Like the second flexibility zone 328 described above with reference to FIG. 6, the transition zone 430 can be formed by removing material from a constant outer diameter tube in order to reduce the outer diameter of a portion of the tube to the third outer diameter WT4c. In at least some cases, the monolithic nature of the transition zone 430 causes it to be stronger than a transition zone made of overlapping tubes welded together. This can reduce or eliminate a benefit from shortening the third portion L1c of the length L1. Accordingly, although the third portion L1c of the length L1 is unchanged in FIG. 7, in other embodiments, a counterpart of the third portion L1c of the length L1 can be longer. In at least some cases, the counterpart of the third portion L1c of the length L1 is at least 2 cm or at least 3 cm. This can help or cause the counterpart of the conduit body 402 to define a variable kink radius, a variable bending stiffness, and/or another variable property.

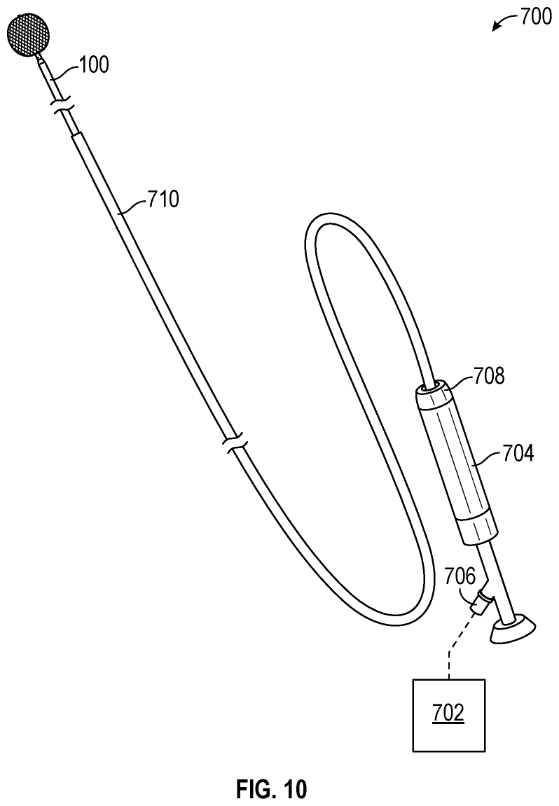

FIG. 8 is a profile view of an embolic material delivery device 500 in accordance with at least some embodiments of the present technology. The embolic material delivery device 500 can be similar to the embolic material delivery device 100 shown in FIG. 1, but with a different configuration of flexibility and transition zones. The embolic material delivery device 500 can include an elongate conduit body 502 defining a length L2 and including a first flexibility zone 526 at a first portion L2a of the length L2, a second flexibility zone 528 at a second portion L2b of the length L2, a first transition zone 530 at a third portion L2c of the length L2 between the first and second portions L2a, L2b of the length L2, a third flexibility zone 538 at a fourth portion L2d of the length L2 distal to the second portion L2b of the length L2, and a second transition zone 540 at a fifth portion L2e of the length L2 between the second and fourth portions L2b, L2d of the length L2. In some cases, the first flexibility zone 526 and the third flexibility zone 538 have the properties discussed above for the first flexibility zone 126 and the second flexibility zone 128, respectively, of the conduit body 102 of the embolic material delivery device 100. In these and other cases, the second flexibility zone 528 can have properties that are intermediate to the respective properties of the first flexibility zone 526 and the third flexibility zone 538. In other embodiments, counterpart conduit bodies can more than just one such intermediate flexibility zone.

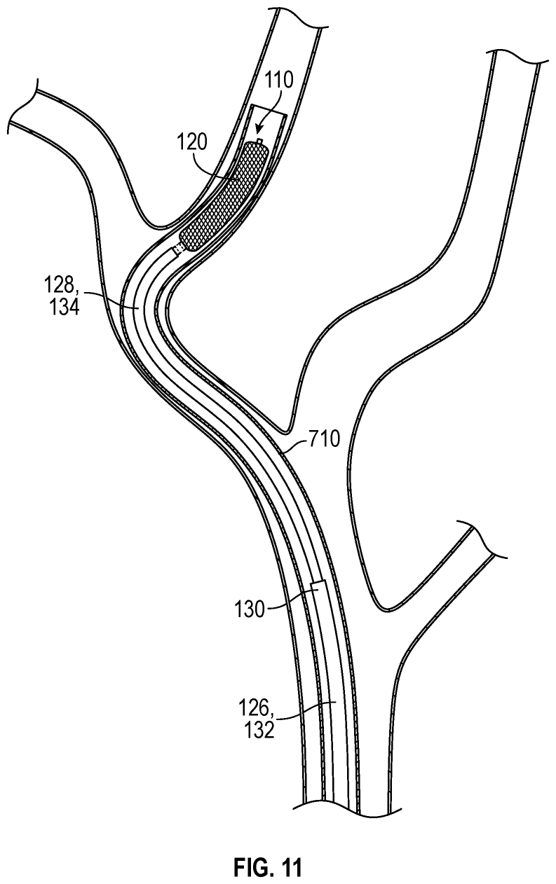

In FIG. 8, the first and second transition zones 530, 540 are shown as dashed boxes to indicate that they can have any of the configurations of the transition zones 130, 230, 330, 430 shown in FIGS. 4-7. FIG. 9 is a longitudinal cross-sectional view of an embolic material delivery device 600 in accordance with at least some embodiments of the present technology corresponding to the embolic material delivery device 500 shown in FIG. 8, but with configurations shown for the transition zones. The embolic material delivery device 600 can include a conduit body 602 that includes a first transition zone 630 and a second transition zone 640 having the configurations of the transition zone 130 and the transition zone 430, respectively. Thus, in some embodiments, one transition zone comprises overlapping tubes and a welded coupling and another comprises a taper between two monolithic flexibility zones. In these embodiments it can be useful for the latter transition zone to be distal to the former transition zone. With reference again to FIG. 9, the configuration of the first transition zone 630 can allow for an advantageous difference in inner diameter at a proximal portion of the conduit body 602 that has relatively little need to bend, while the configuration of the second transition zone 640 allows for advantageous strength and flexibility at a distal portion of the conduit body 602 that does need to move through tortuous anatomy.

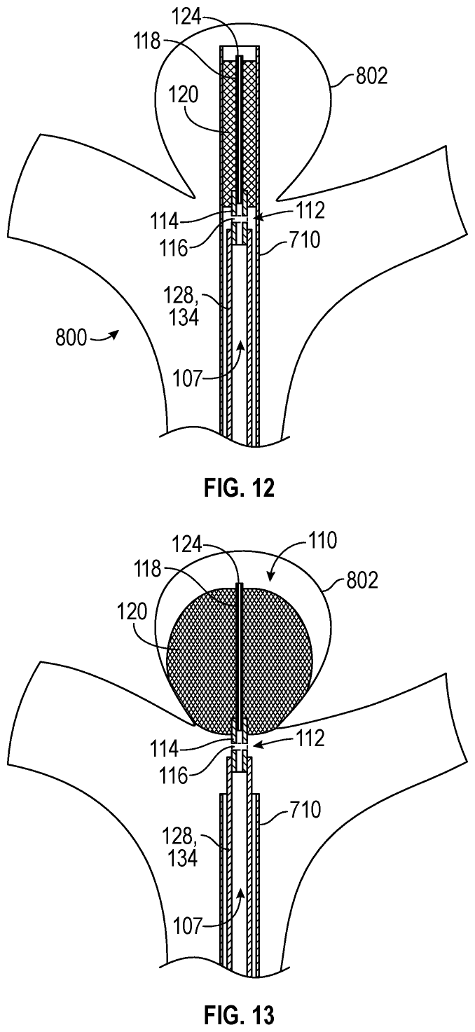

FIG. 10 is a perspective view of the embolic material delivery device 100 and associated structures within a system 700 for treating intracranial aneurysms in accordance with at least some embodiments of the present technology. With reference to FIGS. 1 and 10 together, the system 700 can include a liquid embolic material source 702 and a handle 704 having a port 706 through which the handle 704 is configured to receive liquid embolic material from the source 702. The handle 704 can further include a cap 708 configured to capture the fitting 108. Extending distally from the handle 704, the system 700 can include an elongate sheath 710 slidably connected to the conduit body 102. The embolic material delivery device 100 can be movable relative to the sheath 710 and/or the sheath 710 movable relative to the conduit body 102 such that the expandable structure 110 can be constrained in a low-profile delivery state within the sheath 710 during intravascular delivery and unconstrained to resiliently expand into an expanded state within an aneurysm after intravascular delivery.

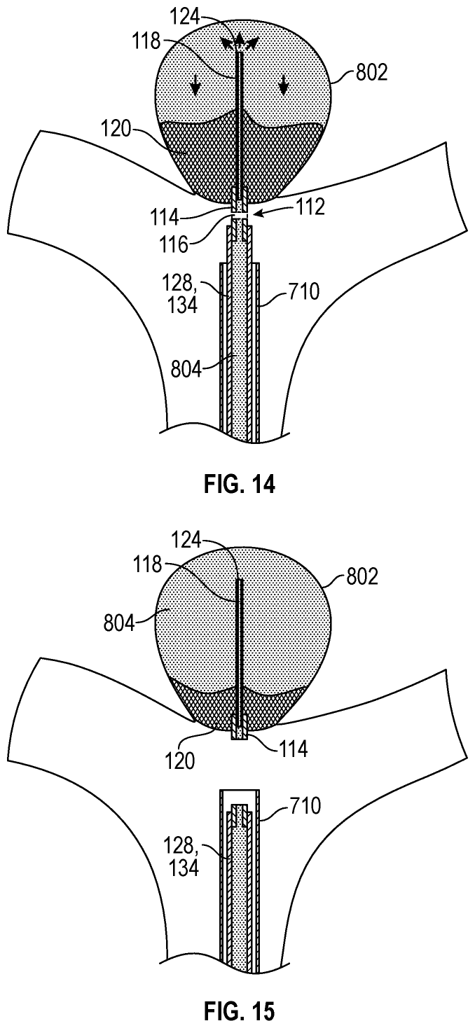

FIGS. 11-15 are longitudinal cross-sectional views of portions of the system 700 and relevant anatomy at different respective times during a method for treating an aneurysm at a neurovascular treatment location in accordance with at least some embodiments of the present technology. As shown in FIG. 11, the method can include moving the distal end portion 106 intravascularly toward the treatment location while the expandable structure 110 is in the delivery state. During this process, the vasculature the second flexibility zone 128 encounters may be more tortuous than the vasculature the first flexibility zone 126 encounters. In at least some cases, moving the distal end portion 106 intravascularly includes bending the second flexibility zone 128 to a radius smaller than a kink radius of the first flexibility zone 126. As discussed above, the first and second flexibility zones 126, 128 can have different kink radiuses. Before, during, and/or after moving the distal end portion 106 intravascularly toward the treatment location, the method can include flowing liquid embolic material distally within and through the first portion of the axial lumen 107 corresponding to the first flexibility zone 126 and then within and through the second portion of the axial lumen 107 corresponding to the second flexibility zone 128. In at least some cases, the liquid embolic material defines a viscosity greater than 30 centistokes. In these and other cases, flowing the liquid embolic material can include overcoming a lower pressure drop per unit length at the first portion of the axial lumen 107 than at the second portions of the axial lumen 107.

FIG. 12 shows portions of the system 700 after the distal end portion 106 reaches a treatment location 800 including an aneurysm 802. The method can include locating the expandable structure 110 at least partially within the aneurysm 802. As shown in FIG. 13, the method can include further retracting the sheath 710 proximally to allow the expandable structure 110 to resiliently expand within the aneurysm 802. The method can further include flowing liquid embolic material 804 distally within the axial lumen 107 and into the aneurysm 802. This can cause the expandable structure 110 to transition from a first state toward a second, more compact state in which the expandable structure 110. For example, force from the liquid embolic material 804 can cause the expandable structure 110 to collapse against a neck of the aneurysm 802 and thereby reduce or prevent leakage of the liquid embolic material 804 from the aneurysm 802. As shown in FIG. 15, when the aneurysm 802 is sufficiently filled, the detachment element 112 can be activated causing it to sever at the detachment zone 116. The embolic material delivery device 100 can then be retracted proximally into the sheath 710. The embolic material delivery device 100 and the sheath 710 can then be removed from the vasculature.

### CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, in alternative embodiments the steps may have another suitable order. Similarly, certain aspects of the present technology disclosed in the context of particular embodiments can be combined or eliminated in other embodiments. Furthermore, while advantages associated with certain embodiments may be disclosed herein in the context of those embodiments, other embodiments may also exhibit such advantages, and not all embodiments need necessarily exhibit such advantages or other advantages disclosed herein to fall within the scope of the present technology. This disclosure and the associated technology can encompass other embodiments not expressly shown or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising,” “including,” and the like are used throughout this disclosure to mean including at least the recited feature(s) such that any greater number of the same feature(s) and/or one or more additional types of features are not precluded. Directional terms, such as “upper,” “lower,” “front,” “back,” “vertical,” and “horizontal,” may be used herein to express and clarify the relationship between various structures. It should be understood that such terms do not denote absolute orientation. Furthermore, reference herein to “one embodiment,” “an embodiment,” or similar phrases means that a particular feature, structure, operation, or characteristic described in connection with such phrases can be included in at least one embodiment of the present technology. Thus, such phrases as used herein are not necessarily all referring to the same embodiment. Finally, it should be noted that various particular features, structures, operations, and characteristics of the embodiments described herein may be combined in any suitable manner in additional embodiments in accordance with the present technology.

I/We claim:

1. An embolic material delivery device comprising:
a conduit body extending between a proximal end portion and a distal end portion, wherein the conduit body defines an axial lumen extending between the proximal end portion and the distal end portion, and wherein the axial lumen is configured to transport the embolic material,
wherein the conduit body comprises a first flexibility zone, a transition zone, and a second flexibility zone,
wherein the first flexibility zone is positioned between the proximal end portion and the transition zone, and wherein the second flexibility zone is positioned between the transition zone and the distal end portion, and
wherein the first flexibility zone is configured to transfer pushing force toward the second flexibility zone and defines a first kink radius, and wherein the second flexibility zone is configured to be navigable through tortuous neurovascular anatomy and defines a second kink radius smaller than the first kink radius.

2. The embolic material delivery device of claim 1, wherein the second kink radius is within a range from 10% to 80% of the first kink radius.

3. The embolic material delivery device of claim 1, further comprising:
a first tube defining a first outer diameter at the first flexibility zone; and
a second tube defining a second outer diameter at the second flexibility zone, wherein the second outer diameter is less than the first outer diameter, and wherein the first tube and the second tube are coupled to one another at the transition zone.

4. The embolic material delivery device of claim 3, further comprising a welded joint coupling the first tube and the second tube to one another at the transition zone.

5. The embolic material delivery device of claim 3, wherein the second tube is positioned within the first tube at the transition zone.

6. The embolic material delivery device of claim 3, wherein the first tube and the second tube are formed from the same material.

7. The embolic material delivery device of claim 6, wherein the second tube is annealed and the first tube is not annealed or is annealed to a lesser extent than the second tube.

8. The embolic material delivery device of claim 6, wherein the first tube and the second tube are nitinol.

9. The embolic material delivery device of claim 1, wherein the first flexibility zone, the second flexibility zone, and the transition zone are a monolithic structure formed from a single piece of material.

10. The embolic material delivery device of claim 9, wherein:
the first flexibility zone defines a first outer diameter; and
the second flexibility zone defines a second outer diameter less than the first outer diameter.

11. The embolic material delivery device of claim 10, wherein the conduit body is formed from a tube defining a constant outer diameter and the second flexibility zone is formed by removing material from the tube in order to reduce the outer diameter of a portion of the tube to the second outer diameter.

12. The embolic material delivery device of claim 10, wherein the transition zone defines a tapering outer diameter transitioning from the first outer diameter to the second outer diameter.

13. The embolic material delivery device of claim 12, wherein the taper extends at least 2 cm in order for the conduit body to define a variable kink radius transitioning from the first kink radius to the second kink radius.

14. The embolic material delivery device of claim 10, wherein the axial lumen defines a constant inner diameter.

15. The embolic material delivery device of claim 1, wherein:
the transition zone is a first transition zone; and
the conduit body comprises:
a third flexibility zone, and
a second transition zone between the second flexibility zone and the third flexibility zone.

16. The embolic material delivery device of claim 15, wherein one of the first transition zone or the second transition zone comprises overlapping tubes and a welded coupling, and the other comprises a taper between two monolithic flexibility zones.

17. The embolic material delivery device of claim 1, further comprising a heat shrink positioned around the first flexibility zone, the transition zone, and the second flexibility zone.

18. A device for treating an intracranial aneurysm, the device comprising:
an elongate conduit body configured to extend intravascularly toward the aneurysm, wherein the conduit body defines an axial lumen through which the conduit body is configured to convey liquid embolic material toward the aneurysm, and wherein the conduit body comprises:
a proximal end portion,
a distal end portion opposite to the proximal end portion along a length of the conduit body,
a first flexibility zone defining a first portion of the length of the conduit body, a first average bending stiffness, and a first outer diameter,
a second flexibility zone defining a second portion of the length of the conduit body, a second average bending stiffness, and a second outer diameter, wherein the second portion of the length of the conduit body is distal to the first portion of the length of the conduit body, wherein the second average bending stiffness is less than the first average bending stiffness, and wherein the second outer diameter is less than the first outer diameter,
a transition zone defining a third portion of the length of the conduit body and a third outer diameter, wherein the third portion of the length of the conduit body is between the first and second portions of the length of the conduit body, and wherein the third outer diameter transitions proximally-to-distally from the first outer diameter to the second outer diameter;
an expandable structure carried by the conduit body, wherein the expandable structure is configured to be disposed at least partially within the aneurysm to reduce leakage of liquid embolic material from the aneurysm; and
a detachment element through which the expandable structure is detachably connected to the conduit body, wherein the detachment element is configured to detach the expandable structure from the conduit body such that the conduit body can be withdrawn intravascularly away from the aneurysm while the expandable structure remains disposed at least partially within the aneurysm.

19. The device of claim 18, wherein the third outer diameter steps down proximally-to-distally from the first outer diameter to the second outer diameter.

20. The device of claim 18, wherein the third outer diameter tapers down proximally-to-distally from the first outer diameter to the second outer diameter.

21. The device of claim 18, wherein the second average bending stiffness is within a range from 5% to 25% of the first average bending stiffness.

22. The device of claim 18, wherein
the first portion of the length of the conduit body is at least 5 cm;
the first outer diameter varies no more than 5% along the first portion of the length of the conduit body;
the second portion of the length of the conduit body is at least 5 cm; and
the second outer diameter varies no more than 5% along the second portion of the length of the conduit body.

23. The device of claim 18, further comprising:
an inlet port operably connected to the axial lumen, wherein the device is configured to receive liquid embolic material via the inlet port, and wherein the transition zone is at least 20 cm distal to the inlet port; and
an outlet port operably connected to the axial lumen, wherein the device is configured to dispense liquid embolic material into the aneurysm via the outlet port, and wherein the transition zone is and at least 20 cm proximal to the outlet port along the length of the conduit body.

24. The device of claim 18, wherein:
the conduit body comprises a tube extending along the first, second, and third portions of the length of the conduit body and defining the first flexibility zone, the second flexibility zone, and the transition zone;
the first flexibility zone, the second flexibility zone, and the transition zone define a first wall thickness, a second wall thickness, and a third wall thickness, respectively;
the first wall thickness is greater than the second wall thickness; and
the third wall thickness decreases proximally-to-distally from the first wall thickness to the second wall thickness.

25. The device of claim 24, wherein:
the first flexibility zone, the second flexibility zone, and the transition zone define a first inner diameter, a second inner diameter, and a third inner diameter, respectively; and
the first, second, and third inner diameters are equal.

26. The device of claim 24, wherein the first flexibility zone defines a pressure rating of at least 20 kpsi.

27. The device of claim 18, wherein:
the conduit body comprises:
a first tube extending along the first and third portions of the length of the conduit body, and
a second tube extending along the second and third portions of the length of the conduit body; and
the second tube overlaps the first tube at the transition zone.

28. The device of claim 27, wherein the first tube comprises a first material, and wherein the second tube comprises a second material compositionally the same as and microstructurally different than the first material.

29. The device of claim 27, wherein:
the first flexibility zone, the second flexibility zone, and the transition zone define a first inner diameter, a second inner diameter, and a third inner diameter, respectively;
the first inner diameter is greater than the second inner diameter; and
the second and third inner diameters are equal.

30. A method for treating an aneurysm at a neurovascular treatment location, the method comprising:
moving a distal end portion of an elongate conduit body intravascularly toward the treatment location, wherein the conduit body defines an axial lumen;
flowing liquid embolic material defining a viscosity greater than 30 centistokes distally within a first portion of the axial lumen, wherein the first portion of the axial lumen defines a first pressure drop per unit length;
flowing the liquid embolic material distally within a second portion of the axial lumen distal to the first portion of the axial lumen, wherein the second portion of the axial lumen defines a second pressure drop per unit length at least 10% less than the first pressure drop per unit length; and
flowing the liquid embolic material into the aneurysm.

31. The method of claim 30, further comprising locating an expandable structure carried by the conduit body at least partially within the aneurysm to reduce leakage of the liquid embolic material from the aneurysm, wherein flowing the liquid embolic material into the aneurysm causes the expandable structure to transition from a first state toward a second state, and wherein the expandable structure occupies less space within the aneurysm in the second state than in the first state.

32. The method of claim 30, wherein moving the distal end portion intravascularly toward the treatment location comprises bending a second flexibility zone of the conduit body to a radius smaller than a kink radius of a first flexibility zone of the conduit body, wherein the first flexibility zone defines the first portion of the axial lumen, and wherein the second flexibility zone defines the second portion of the axial lumen.
